(19) United States
(12) Patent Application Publication
(10) Pub. No.: US 2019/0257803 A1
(43) Pub. Date: Aug. 22, 2019

(54) GAS SENSOR BASELINE CORRECTION USING MULTIPLE CO-LOCATED GAS SENSORS

(71) Applicant: Apple Inc., Cupertino, CA (US)

(72) Inventors: Michael K. BROWN, Sunnyvale, CA (US); Roham SOLASI, San Francisco, CA (US); Miaolei YAN, Santa Clara, CA (US)

(21) Appl. No.: 16/259,915

(22) Filed: Jan. 28, 2019

Related U.S. Application Data
(60) Provisional application No. 62/634,134, filed on Feb. 22, 2018.

(51) Int. Cl. G01N 33/00; G01N 27/12

(52) U.S. Cl. CPC G01N 33/0006; G01N 27/125; G01N 27/123; G01N 33/0073; G01N 33/0031; G01N 33/0067; G01N 33/0016

(57) ABSTRACT

A portable communication device includes a first gas sensor, one or more second gas sensors, at least one port and a calibration module. The first gas sensor is disposed on a first hotplate and is enclosed in an enclosure. The one or more second gas sensors are disposed on one or more second hotplates and are also enclosed in the enclosure. The ports can allow flow of air into and out of the enclosure. The second gas sensors are stable gas sensors, which are used by the calibration module to calibrate the first gas sensor.

### CROSS-REFERENCE TO RELATED APPLICATIONS

The present application claims the benefit of U.S. Provisional Patent Application Ser. No. 62/634,134, entitled “Gas Sensor Baseline Correction Using Multiple Co-Located Gas Sensors” filed on Feb. 22, 2018, which is hereby incorporated by reference in its entirety for all purposes.

### TECHNICAL FIELD

The present description relates generally to transducers, and more particularly, to gas sensor baseline correction using multiple co-located gas sensors.

### BACKGROUND

Miniature gas sensors for consumer electronics represent a technology category that could enable upcoming features and/or products in applications such as environmental and health monitoring, smart homes, and internet of things (IoT). Metal oxide (MOX) gas sensors are among the most promising technologies to be integrated with consumer electronic devices due to their small size, low power consumption, compatibility with semiconductor fabrication processes, and relatively simple architecture. Chemical poisoning and deactivation of the sensor materials in metal oxide sensors, however, can cause drift in both baseline resistance and sensitivity, which can pose great challenges to the mass market adoption of miniature gas sensors.

Many MOX gas sensors consist of a porous MOX material disposed on a micro-hotplate, which is used to regulate temperature. When heated to the working temperature, the resistance of the metal oxide material changes with the gas environment and concentration. The target gas can be an oxidizing gas such as ozone (O₃) or nitrogen oxide (NOₓ), which increases MOX resistance. The target gas may be a reducing gas, for example, hydrogen (H₂) or volatile organic compounds (VOC), which decrease the MOX resistance.

### BRIEF DESCRIPTION OF THE DRAWINGS

Certain features of the subject technology are set forth in the appended claims. However, for purposes of explanation, several embodiments of the subject technology are set forth in the following figures.

FIG. 1 is a schematic diagram illustrating example gas-sensing devices using co-located gas sensors for baseline calibration, in accordance with one or more aspects of the subject technology.

FIG. 2 is a schematic diagram illustrating example gas-sensing devices using co-located gas sensors for baseline calibration, in accordance with one or more aspects of the subject technology.

FIGS. 3A and 3B are flow diagrams illustrating example processes for calibrating a gas sensor using a co-located gas sensor, in accordance with one or more aspects of the subject technology.

FIGS. 4A, 4B and 4C are charts illustrating example plots of accumulated drifts of a sensor and a stable-baseline co-located sensor, in accordance with one or more aspects of the subject technology.

FIG. 5 is a flow diagram illustrating a method of providing the gas-sensing device of FIG. 1, in accordance with one or more aspects of the subject technology.

FIG. 6 is a block diagram illustrating an example wireless communication device, within which one or more miniature gas sensors of the subject technology can be integrated.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced without one or more of the specific details. In some instances, structures and components are shown in block diagram form in order to avoid obscuring the concepts of the subject technology.

In one or more aspects, the subject technology is directed to gas sensor baseline correction using multiple co-located gas sensors. In some embodiments, the gas sensor to be calibrated and the co-located gas sensors can be, but are not limited to, metal oxide (MOX) gas sensors. MOX gas sensors often consist of a porous MOX material dispensed on a micro-hotplate, which is used for regulating the temperature of the gas sensor. The MOX gas sensors can be calibrated in known gas environments, for example, at zero target gas concentration. The calibration allows establishing the MOX resistance characteristics at varying levels of target gas. The target gas concentration can be inferred from the measured MOX resistance values corrected using the subject calibration technique. The calibration technique of the subject technology can be performed at various times, for example, periodically and at different locations (e.g., indoors and outdoors). In other words, the device need not be in a specific location to be calibrated.

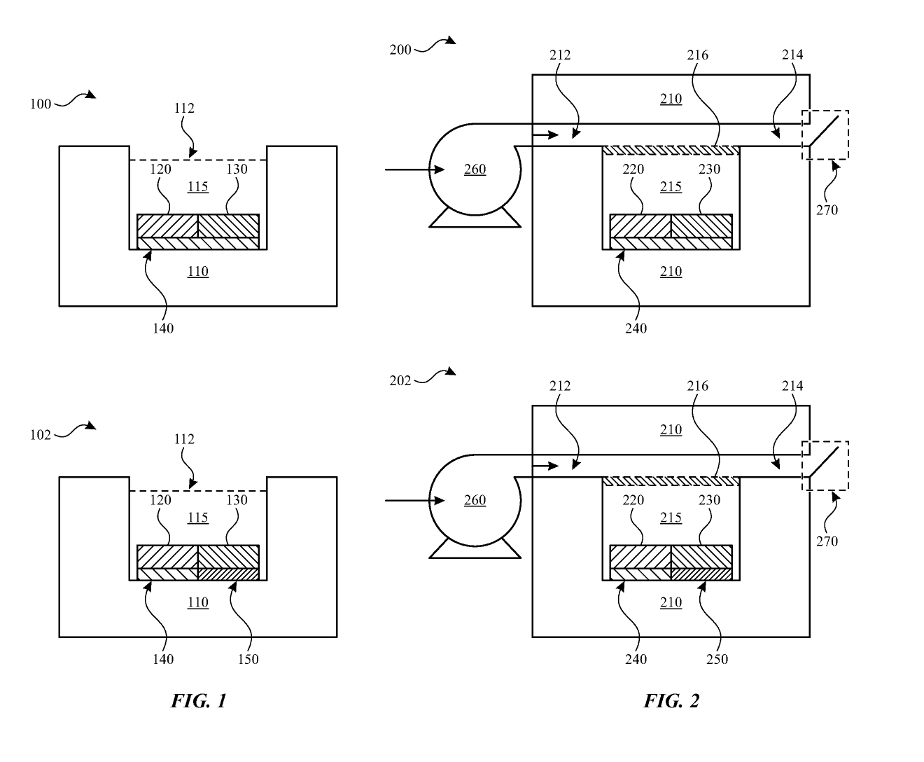

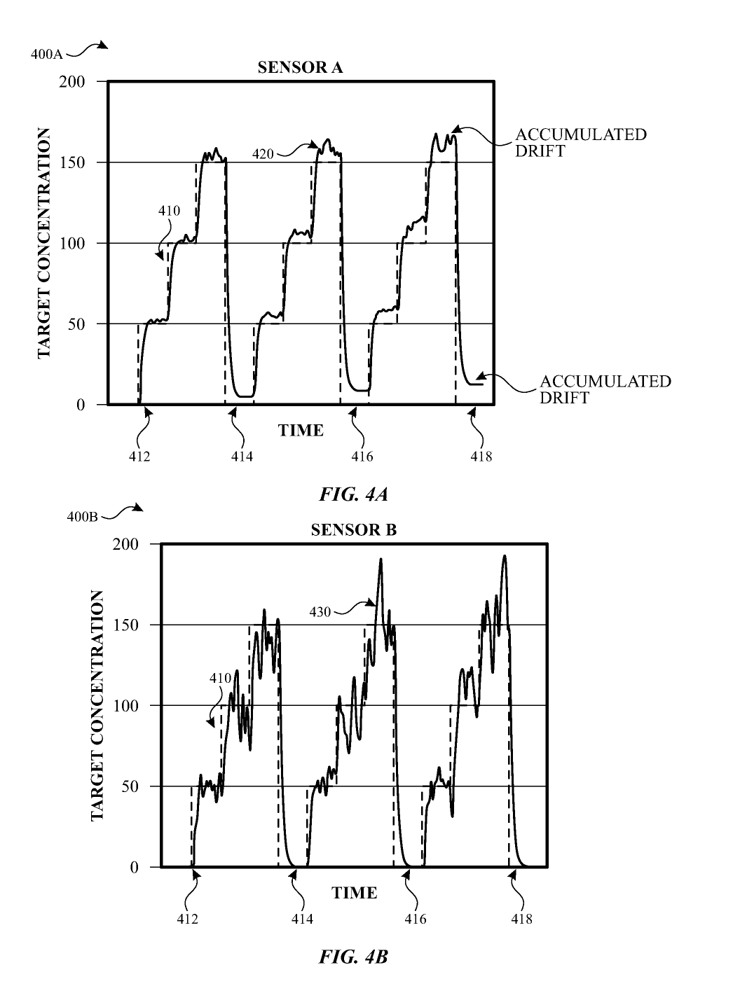

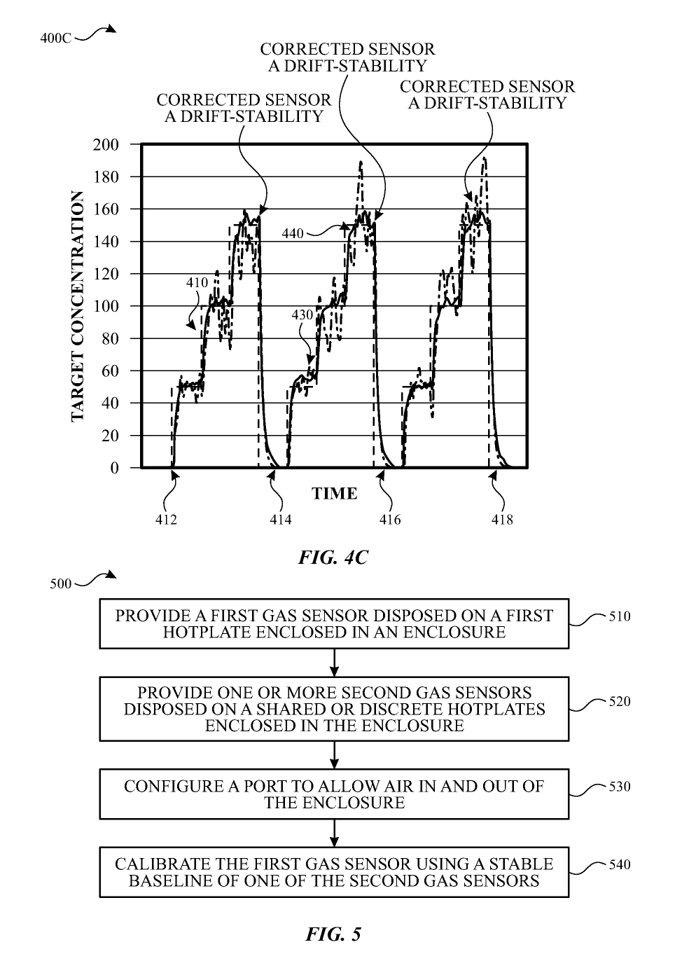

FIG. 1 is a schematic diagram illustrating example gas-sensing devices 100 and 102 using co-located gas sensors for baseline calibration, in accordance with one or more aspects of the subject technology. The gas sensing devices 100 and 102 can be integrated with a host device such as a consumer electronic device, for example, a portable communication device (e.g., a smart phone or a smart watch). The gas-sensing device 100 includes an enclosure 110 including an air permeable port 112, a hotplate 140, a first gas sensor 120 and a co-located gas sensor 130. In some implementations, multiple co-located gas sensors may be used. The air including a gas of interest (e.g., a target gas) such as ozone, VOC, methane, carbon oxides (e.g., COₓ), nitrogen oxides (e.g., NOₓ) or another gas of interest can enter a cavity 115 of the gas-sensing device 100 through the air permeable port 112 and be sensed by the first gas sensor 120 and the co-located gas sensor 130. The first gas sensor 120 and the co-located gas sensor 130 are sensitive to the gas of interest and the gas-sensing device 100 can characterize the gas of interest, for example, by measuring a concentration of the gas of interest. The sensitivity of the first gas sensor 120 and the co-located gas sensor 130 to the target gas is characterized by their resistivity, which can change with variation of concentration of the target gas (as shown in FIG. 4A).

In some implementations, the first gas sensor 120 and the co-located gas sensor 130 can be metal oxide (MOX) sensors, but are not limited to the MOX sensors and can be made of other appropriate gas sensitive materials suitable for sensing the target gas. The underlying principle of MOX gas sensors are based on chemisorption of oxidizing or reducing gas species on the oxide surface, which is followed by a charge transfer process that can result in resistance changes of the MOX material. Examples of metal oxide materials include, but are not limited to, tin oxide (SnO₂), tungsten oxide (WO₃), indium oxide (In₂O₃), zinc oxide (ZnO) or such base materials doped with electronic or chemical catalysts including platinum (Pt), gold (Au), palladium (Pd), silver (Ag), and the like.

In some embodiments as shown in the gas-sensing device 100, the first gas sensor 120 and the co-located gas sensor 130 can be formed (e.g., deposited) on the same hotplate 140. In some implementations, the co-located gas sensor 130 can be formed on separate hotplates 150, as shown in the gas-sensing device 102. The hotplates 140 and 150 can be micro electromechanical system (MEMS) hotplates and can include titanium nitride, which is compatible with complementary metal-oxide semiconductor (CMOS) process and has a high melting point (e.g., 2950° C.), although other suitable metals may be used. In some implementations, the hotplates 140 and 150 can be controlled (e.g., by a microcontroller or a general processor) and can be used to regulate the temperature of the first gas sensor 120 and the co-located gas sensor 130.

The first gas sensor 120 may have drifting characteristics (e.g., sensitivity) that may change over time and cannot be practically recalibrated without a known gas environment or co-located reference instrument. Drift is defined as a small, non-deterministic temporal variations of the sensor response to the analyte under the same condition. Drift may result in either overestimation or underestimation of the target gas concentration over time. An important feature of the co-located gas sensor 130 of the subject technology is a stable characteristic (sensitivity), for example, a stable baseline sensitivity (e.g., at zero concentration of the gas of interest), based on which calibration of the first gas sensor 120 is performed.

The co-located gas sensor 130 does not need to be a perfect sensor in terms of noise and other aspects and can be a rather poor sensor with a stable baseline sensitivity. The zero concentration of the target gas may be achieved, for example, by changing the environment that the gas-sensing device 100 is exposed to. For instance, moving from an outdoor environment where certain gas species (e.g., the target gas) are prevalent to an indoor environment, in which the target gas is known to not exist. The stable characteristics of sensor 130 could be attributed to MOX gas sensors of a different chemical composition, morphology, microstructure and/or processing conditions. The disclosed calibration techniques do not rely on the calibration performed in the factory and allows multiple (e.g., periodic) in-field calibrations of the gas-sensing device (e.g., 100 or 102). In one or more implementations, the material for the enclosure 110 may be a metal such as aluminum, stainless steel or other metals or metallic alloys or other suitable materials. The air permeable port 112 may be a membrane made of a porous material, which can be waterproof to protect the sensors against moisture and humidity.

FIG. 2 is a schematic diagram illustrating example gas-sensing devices 200 and 202 using co-located MOX sensors for baseline calibration, in accordance with one or more aspects of the subject technology. The gas sensing devices 200 and 202 can be integrated with a host device such as a consumer electronic device, for example, a portable communication device (e.g., a smart phone or a smart watch). The gas-sensing device 200 includes an enclosure 210 including an inlet port 212 and an outlet port 214, a hotplate 240, a first gas sensor 220 and a co-located gas sensor 230. In one or more implementations, the gas-sensing device 200 includes an air-permeable membrane 216 that can be made of a waterproof porous material to protect the first gas sensor 220 and the co-located gas sensor 230 from moisture and humidity.

In some implementations, multiple co-located gas sensors may be used. The material for the first gas sensor 220, the co-located gas sensor 230 and the hotplate 240 are similar to the first gas sensor 120, the co-located gas sensor 130 and the hotplate 140 of FIG. 1. The inlet port 212 may include an air blower 260, and the outlet port 214 may include a valve 270. The air blower 260 may be a mechanical pump, a piezo pump or a speaker of a host device. In one or more implementations, the valve 270 can be a one-way valve that only allows air to exhaust from a cavity 215 of the gas-sensing device 200. The air blower 260 and the valve 270 may be controlled by a processor (e.g., a microcontroller or a general-purpose processor) such as a processor of the host device. The air blower 260 and the valve 270 may be used to established different amounts of air, and as a result, different concentrations of the target gas in the cavity 215. In some implementations, the location of air blower 260 and the valve 270 can be interchanged. For example, a one-way valve that can prohibit gas escape from the cavity 215 can be used at the inlet port 212, and an air blower capable of sucking the gas from the cavity 215 may be used at the outlet port 214.

The air including a target gas such as ozone, VOC, methane, carbon oxides (e.g., COₓ), nitrogen oxides (e.g., NOₓ) or another target gas can enter a cavity 215 of gas-sensing device 200 via the inlet port 212 and be sensed by the first gas sensor 220 and the co-located gas sensor 230. The first gas sensor 220 and the co-located gas sensor 230 are sensitive to the target gas and the gas-sensing device 200 can measure, for example, a concentration of the gas of interest.

The gas-sensing device 202 is similar to the gas-sensing device 200, except that the co-located gas sensor 230 is formed on a separate hotplate 250. The temperatures of hotplates 240 and 250 can be independently controlled (e.g., by a processor).

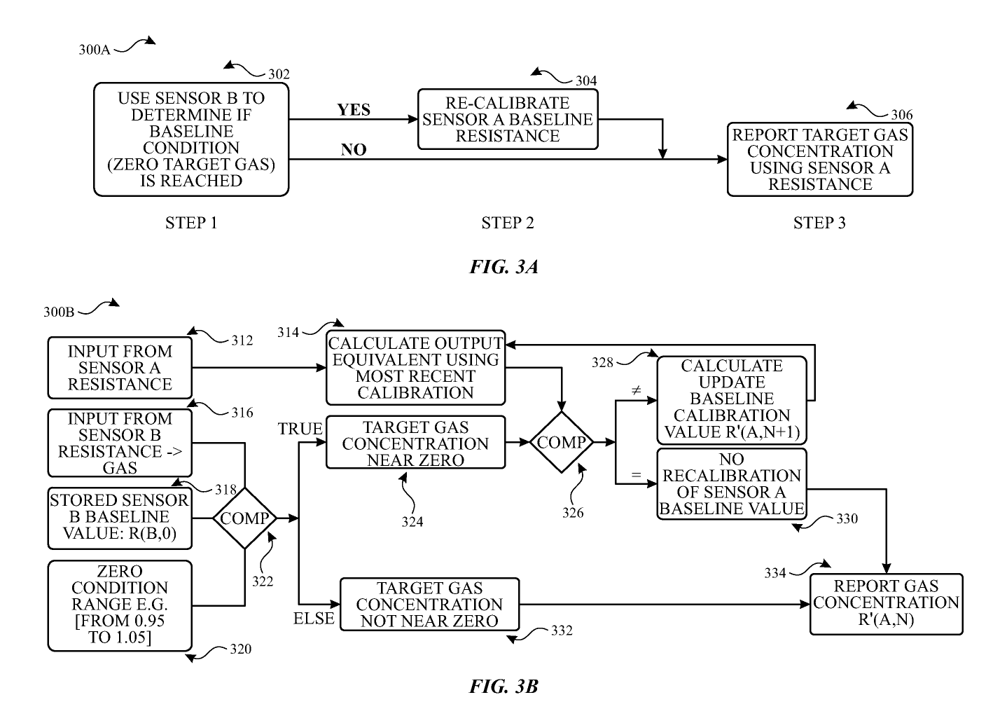

FIGS. 3A and 3B are flow diagrams illustrating example processes 300A and 300B for calibrating a gas sensor using a co-located sensor, in accordance with one or more aspects of the subject technology. The processes 300A and 300B may be used to calibrate the first gas sensor (also referred to as “sensor A”) such as 120 of FIG. 1 or 220 of FIG. 2, using respective co-located gas sensors (also referred to as “sensor B”), for example, 130 of FIG. 1 or 230 of FIG. 2. The process 300A is a high-level process that is controlled by a calibration module of a processor (e.g., a microcontroller or a processor) of the host device. The processes 300A starts at operation block 302 (step 1), where a resistance of the co-located gas sensor 130 (sensor B) is measured to obtain the target gas concentration and to determine whether the baseline condition (e.g., zero concentration of the target gas) is reached. If the baseline condition is reached, at operation block 304 (step 2), the baseline resistance of the first gas sensor 120 (sensor A) is recalibrated. Otherwise, if the baseline condition is not reached, or after completion of step 2, the control is passed to operation block 306, where the target gas concentration is measured by the first gas sensor 120 (sensor A) and is reported (e.g., to the processor).

The process 300B includes more detailed steps of the high-level process 300A and is also controlled by the calibration module of the processor of the host device. The cavity of the gas-sensing device (e.g., 100 of FIG. 1 or 200 of FIG. 2) contains the target gas. The processes 300B starts at operation block 312, where a resistance of the sensor A (e.g., first gas sensor 120 or 220) is measured. At operation block 314, the measured resistance of the first gas sensor 120 is converted to a target gas concentration based on the most recent calibration. At operation block 316, a resistance of the sensor B (e.g., co-located gas sensor 130 or 230) is measured, and at control operation block 322, is compared with a stored baseline value 318 of the sensor B (e.g., R(b,0)). In comparison made in the control operation block 322, the measured sensor B resistance is actually compared with a zero condition range that is within a range of about 0.95 to 1.05 of R(b,0), as shown in block 320. If the measured sensor B shows a near zero target gas concentration (e.g., within a range of about 0.95 to 1.05 of R(b,0), at operation block 326, the near zero target gas concentration value of block 324 is compared with the target gas concentration result of operation block 314. If the near zero target gas concentration value of block 324 is equal to the target gas concentration result of operation block 314, control is passed to operation block 330, where no recalibration of the baseline value of sensor A is performed and, at operation block 334, target gas concentration result of operation block 314 is reported as the current target gas concentration. The operation block 334 is also reached if the measured sensor B, at control operation block 322, does not show a near zero target gas concentration (e.g., within a range of about 0.95 to 1.05 of R(b,0)) (332). If the near zero target gas concentration value 324 is not equal to the target gas concentration result of operation block 314, control is passed to operation block 328, where an updated baseline calibration value R′(a, n+1) is determined and reported to be used in operation block 314.

In summary, the updated baseline calibration value R′(a, n+1) is calculated if the sensor B, which has a stable baseline value, shows a near zero target gas concentration and the calculated target gas concentration (e.g., in 314) based on sensor A is different form the near zero target gas concentration.

Additionally, the baseline detection can be improved by, for example, integration of the gas-sensing device (e.g., 100 of FIG. 1) with a relative humidity and temperature sensor, the output of which can be utilized to correct for impacts of these conditions on the measured baseline resistance. In some implementations, the baseline detection can be improved by integration with reducing gas (e.g. VOCs) sensors to detect real events and reject false baseline detections. In one or more implementations, other features, such as indoor/outdoor detection, water occlusion detection and the like can be integrated with the gas-sensing device of the subject technology to improve confidence of zero gas concentration (baseline) measurements, for example, by sensors A and/or B.

FIGS. 4A, 4B and 4C are charts 400A, 400B and 400C illustrating example plots of accumulated drifts of a sensor and a stable-baseline co-located sensor, in accordance with one or more aspects of the subject technology. The chart 400A shows a plot 410 of a few cycles of a target gas concentration versus time. In each cycle, the target gas concentration passes through a number of steps while rising and suddenly drops to zero concentration (e.g., at times 412, 414, 416 and 418). The plot 420 shows a resistance characteristic of the sensor A (e.g., 120 of FIG. 1), indicating that the variation of the resistance values of the sensor A versus time trends well with the plot 410, particularly in a first cycle, but some drift is observed in baseline values (e.g., at times 414, 416 and 418). As shown by the plot 420, the drifts in the baseline values (e.g., zero concentration of the target gas) are accumulative and tend to increase with time. This results in increasingly large measurement errors at both baseline and at higher target gas concentrations values. The subject technology uses co-located gas sensors (e.g., sensor B, such as 130 of FIG. 1) to calibrate the sensor A, as needed, based on a stable co-located gas sensor B (e.g., 130 of FIG. 1).

The chart 400B shows the plot 410 of the target gas concentration versus time and a plot 430 of the resistance characteristic of sensor B, exposed to the target gas. The interesting feature of the sensor B is a stable baseline which is observed at zero target gas concentrations (e.g., at times 412, 414, 416 and 418). The plot 420 shows that the sensor B, other than having a desirable stable baseline resistance, in general, does not trend variations of the target gas concentration with time as well as the sensor A does. In other words, the sensor B appears to be a noisy sensor and with an inferior performance in reporting elevated levels (span) of the target gas, but can accurately detect zero (or near zero) target gas events. The baseline resistance (RO) for Sensor B can be measure at zero gas concentration (To) and stored for reference in a memory of the host device. Subsequent zero gas concentration detection can be enabled by comparing to Ro value, as described below.

The chart 400C shows the plot 410 of the target gas concentration versus time, the plot 430 of the resistance characteristic of sensor B and a corrected characteristic 440 of sensor A using the zero target gas concentration detection by sensor B. When a zero target gas event is accurately detected by sensor B, a resistance Ro value of sensor B is used to re-calibrate the zero target gas baseline of sensor A, thus eliminating the accumulated drift of sensor A as shown in FIG. 400B. The subject approach allows the gas sensor A (e.g., an MOX sensor), which is prone to baseline drift, to be utilized and normalized against long-term accumulated baseline error.

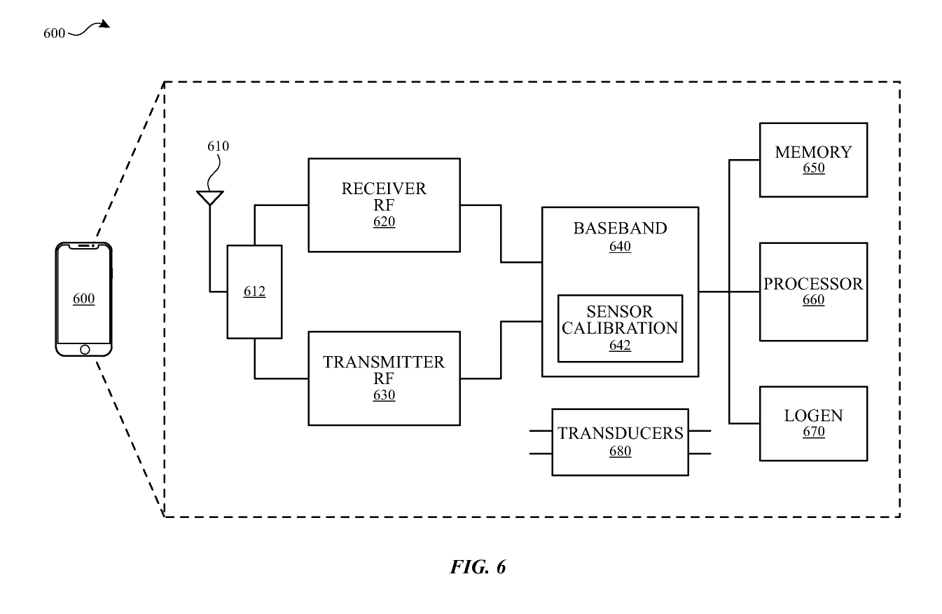

FIG. 5 is a flow diagram illustrating a method 500 of providing of the gas-sensing device 100 of FIG. 1, in accordance with one or more aspects of the subject technology. The method 500 starts with providing a first gas sensor (e.g., 120 of FIG. 1) disposed on a hotplate (e.g., 140 of FIG. 1) enclosed in an enclosure (e.g., 110 of FIG. 1) (510). One or more second gas sensors (e.g., 130 of FIG. 1) may be disposed on a shared or discrete hotplate (e.g., 140 or 150 of FIG. 1) enclosed in the enclosure (520). A port (e.g., 112 of FIG. 1) may be configured to allow air in and out of the enclosure (530). The first gas sensor may be calibrated (e.g., by the 642 of FIG. 6) using a stable baseline (see 412, 414, 416 and 416 of FIG. 4B) of one of the second gas sensors (540).

FIG. 6 is a block diagram illustrating an example wireless communication device 600, within which one or more miniature gas sensors of the subject technology can be integrated. The wireless communication device 600 may comprise a radio-frequency (RF) antenna 610, a receiver 620, a transmitter 630, a baseband processing module 640, a memory 650, a processor 660, a local oscillator generator (LOGEN) 670 and one or more transducers 680. In various embodiments of the subject technology, one or more of the blocks represented in FIG. 6 may be integrated on one or more semiconductor substrates. For example, the blocks 620-670 may be realized in a single chip or a single system on a chip, or may be realized in a multi-chip chipset.

The receiver 620 may comprise suitable logic circuitry and/or code that may be operable to receive and process signals from the RF antenna 610. The receiver 620 may, for example, be operable to amplify and/or down-convert received wireless signals. In various embodiments of the subject technology, the receiver 620 may be operable to cancel noise in received signals and may be linear over a wide range of frequencies. In this manner, the receiver 620 may be suitable for receiving signals in accordance with a variety of wireless standards, Wi-Fi, WiMAX, Bluetooth, and various cellular standards. In various embodiments of the subject technology, the receiver 620 may not require any SAW filters and few or no off-chip discrete components such as large capacitors and inductors.

The transmitter 630 may comprise suitable logic circuitry and/or code that may be operable to process and transmit signals from the RF antenna 610. The transmitter 630 may, for example, be operable to up-convert baseband signals to RF signals and amplify RF signals. In various embodiments of the subject technology, the transmitter 630 may be operable to up-convert and amplify baseband signals processed in accordance with a variety of wireless standards. Examples of such standards may include Wi-Fi, WiMAX, Bluetooth, and various cellular standards. In various embodiments of the subject technology, the transmitter 630 may be operable to provide signals for further amplification by one or more power amplifiers.

The duplexer 612 may provide isolation in the transmit band to avoid saturation of the receiver 620 or damaging parts of the receiver 620, and to relax one or more design requirements of the receiver 620. Furthermore, the duplexer 612 may attenuate the noise in the receive band. The duplexer may be operable in multiple frequency bands of various wireless standards.

The baseband processing module 640 may comprise suitable logic, circuitry, interfaces, and/or code that may be operable to perform processing of baseband signals. The baseband processing module 640 may, for example, analyze received signals and generate control and/or feedback signals for configuring various components of the wireless communication device 600, such as the receiver 620. The baseband processing module 640 may be operable to encode, decode, transcode, modulate, demodulate, encrypt, decrypt, scramble, descramble, and/or otherwise process data in accordance with one or more wireless standards. In some implementations, the baseband processing module 640 may include a sensor calibration module 642 that can control calibration processes (e.g., 300A and 300B of FIG. 3) of the miniature gas sensor of the subject technology.

The processor 660 may comprise suitable logic, circuitry, and/or code that may enable processing data and/or controlling operations of the wireless communication device 600. In this regard, the processor 660 may be enabled to provide control signals to various other portions of the wireless communication device 600. The processor 660 may also control transfers of data between various portions of the wireless communication device 600. Additionally, the processor 660 may enable implementation of an operating system or otherwise execute code to manage operations of the wireless communication device 600. In one or more implementations, the processor 660 may control calibration processes (e.g., 300A and 300B of FIG. 3) of the miniature gas sensor of the subject technology.

The memory 650 may comprise suitable logic, circuitry, and/or code that may enable storage of various types of information such as received data, generated data, code, and/or configuration information. The memory 650 may comprise, for example, RAM, ROM, flash, and/or magnetic storage. In various embodiment of the subject technology, information stored in the memory 650 may be utilized for configuring the receiver 620 and/or the baseband processing module 640.

The local oscillator generator (LOGEN) 670 may comprise suitable logic, circuitry, interfaces, and/or code that may be operable to generate one or more oscillating signals of one or more frequencies. The LOGEN 670 may be operable to generate digital and/or analog signals. In this manner, the LOGEN 670 may be operable to generate one or more clock signals and/or sinusoidal signals. Characteristics of the oscillating signals such as the frequency and duty cycle may be determined based on one or more control signals from, for example, the processor 660 and/or the baseband processing module 640.

In operation, the processor 660 may configure the various components of the wireless communication device 600 based on a wireless standard according to which it is desired to receive signals. Wireless signals may be received via the RF antenna 610 and amplified and down-converted by the receiver 620. The baseband processing module 640 may perform noise estimation and/or noise cancellation, decoding, and/or demodulation of the baseband signals. In this manner, information in the received signal may be recovered and utilized appropriately. For example, the information may be audio and/or video to be presented to a user of the wireless communication device, data to be stored to the memory 650, and/or information affecting and/or enabling operation of the wireless communication device 600. The baseband processing module 640 may modulate, encode, and perform other processing on audio, video, and/or control signals to be transmitted by the transmitter 630 in accordance with various wireless standards.

The one or more transducers 680 may include a miniature gas sensor of the subject technology as shown in FIGS. 1 and 2 and described above. The miniature gas sensor of the subject technology can be readily integrated into the communication device 600, in particular when the communication device 600 is a smart mobile phone or a smart watch.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as a “configuration” does not imply that such configuration is essential to the subject technology or that such configuration applies to all configurations of the subject technology. A disclosure relating to a configuration may apply to all configurations, or one or more configurations. A phrase such as a configuration may refer to one or more configurations and vice versa.

The word “example” is used herein to mean “serving as an example or illustration.” Any aspect or design described herein as “example” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A portable communication device, the device comprising:
a first gas sensor disposed on a first hotplate and enclosed in an enclosure;
one or more second gas sensors disposed on one or more second hotplates and enclosed in the enclosure;
at least one port configured to allow flow of air into and out of the enclosure; and
a calibration module,
wherein the first gas sensor and the one or more second gas sensors are sensitive to a target gas, and the calibration module is configured to calibrate the first gas sensor for the target gas using a stable baseline of the one or more second gas sensors.

2. The device of claim 1, wherein the first gas sensor comprises a miniature gas sensor including a metal-oxide (MOX) gas sensor.

3. The device of claim 1, wherein the one or more stable gas sensors comprise miniature gas sensors that feature stable baselines including MOX gas sensors of a different chemical composition, morphology, microstructure or processing conditions.

4. The device of claim 1, wherein the one or more second gas sensors are disposed side-by-side with the first gas sensor.

5. The device of claim 4, wherein the first hotplate and the one or more second hotplates are configured to be controlled independent of one another.

6. The device of claim 4, wherein a common hotplate is used to replace the one or more second hotplates and the first hotplate, and wherein the common hotplate is configured to simultaneously regulate temperatures of the first gas sensor and the one or more second gas sensors.

7. The device of claim 1, wherein the at least one port includes an air-permeable membrane.

8. The device of claim 1, wherein the at least one port comprises an inlet or an outlet port including an air blower.

9. The device of claim 8, further comprising an air-permeable membrane, and wherein the air blower comprises a mechanical pump, a piezo pump or a speaker of the device.

10. The device of claim 8, wherein the at least one port further comprises an outlet port or an inlet port including a one-way valve configured to allow adjusting gas content of the enclosure by controlling flow of air in or out of the enclosure.

11. A device comprising:
an enclosure including at least one port;
one or more hotplates enclosed in the enclosure;
a first gas sensor disposed on one of the one or more hotplates; and
at least one second gas sensor disposed on the one or more hotplates,
wherein the at least one port is configured to permit flow of air into and out of the enclosure, and the at least one second gas sensor is sensitive to a target gas and is stable over at least a range of concentration values of the target gas and is used to calibrate the first gas sensor for measuring concentration values of the target gas.

12. The device of claim 11, wherein the first gas sensor and the at least one of the second gas sensors are disposed on a common hotplate that is configured to regulate temperatures of the first gas sensor and the at least one of the second gas sensors simultaneously.

13. The device of claim 11, wherein the first gas sensor and the at least one of the second gas sensors are disposed on separate hotplates that are configured to regulate temperatures of the first gas sensor and the at least one of the second gas sensors independently.

14. The device of claim 11, wherein the at least one port is covered with an air-permeable membrane that allows gas content of the enclosure be in equilibrium with an environment surrounding the device.

15. The device of claim 11, wherein the at least one port includes an inlet port and an outlet port.

16. The device of claim 15, wherein a blower is used to draw air through the inlet port and the outlet port includes a controllable valve.

17. The device of claim 16, wherein the blower comprises one of a mechanical pump, a piezo pump or a speaker of the device, and wherein the controllable valve comprises a one-way valve configured to prohibit air from entering the enclosure from the outlet port.

18. The device of claim 11, wherein the at least one second gas sensor has a stable baseline, and wherein the device further comprises a calibration module configured to periodically calibrate the first gas sensor based on the at least one second gas sensor.

19. A system comprising:
a communication device;
a miniature gas sensor and one or more calibration gas sensors integrated with the communication device; and
a calibration processor,
wherein the calibration processor is configured to calibrate the miniature gas sensor for a target gas based on a reading of at least one the one or more calibration gas sensors,
at least one of the one or more calibration gas sensors are sensitive to the target gas and features a stable baseline, and the miniature gas sensor and one or more calibration gas sensors are disposed on one or more hotplates.

20. The system of claim 19, wherein the miniature gas sensor and one or more calibration gas sensors are enclosed in an enclosure including at least one port, wherein the at least one port is covered by an air-permeable membrane or includes an inlet port and an outlet port, and wherein one of the inlet port or the outlet port includes an air blower and another one of the inlet port and the outlet port includes a one-way valve.
